(19) United States
(12) Patent Application Publication
(10) Pub. No.: US 2009/0081773 A1
(43) Pub. Date: Mar. 26, 2009

(54) MICROFLUIDIC APPARATUS FOR MANIPULATING IMAGING AND ANALYZING CELLS OF A CYTOLOGICAL SPECIMEN

(75) Inventor: Howard Kaufman, Newton, MA (US)

Correspondence Address: VISTA IP LAW GROUP LLP, 12930 Saratoga Avenue, Suite D-2, Saratoga, CA, 95070, US

(73) Assignee: CYTYC CORPORATION, Marlborough, MA (US)

(21) Appl. No.: 12/235,470

(22) Filed: Sep. 22, 2008

Related U.S. Application Data
(60) Provisional application No. 60/975,070, filed on Sep. 25, 2007.

(51) Int. Cl. G01N 21/00; G01N 33/487; G01N 1/28

(52) U.S. Cl. 435/309.1; 422/101

(57) ABSTRACT

A microfluidic apparatus for isolating and imaging or analyzing cells of a cytological specimen includes a substrate and a microfluidic cellular isolation element that includes an outer wall, a channel, a partition member and a receptacle. The partition member is positioned within the isolation element interior, and the receptacle is positioned within the partition member interior. The isolation element is configured such that fluid introduced through the outer wall inlet flows through the channel in a first direction, and the partition member is situated such that fluid flows from the channel into the partition member interior through the partition member inlet aperture in a second direction different than the first direction. The receptacle positioned relative to the partition member inlet to catch and retain a cell carried by the fluid.

### RELATED APPLICATION DATA

The present application claims the benefit under 35 USC §119 of provisional application Ser. No. 60/975,070, filed Sep. 25, 2007. The aforementioned application is hereby incorporated by reference herein in its entirety.

### FIELD OF THE INVENTION

The field of the invention relates to processing biological specimens, and more particularly, to isolating and imaging cells of a biological specimen using microfluidics devices.

### BACKGROUND

Medical professionals and technicians often prepare a biological specimen on a specimen carrier, such as a glass specimen slide, and review the specimen to analyze whether a patient has or may have a particular medical condition or disease. For example, a specimen is examined to detect malignant or pre-malignant cells as part of a Papanicolaou (Pap) smear test and other cancer detection tests. After a specimen slide has been prepared, automated systems can analyze the specimen and be used to focus the technician's attention on the most pertinent cells or groups of cells, while discarding less relevant cells from further review.

While Pap smears are well known, they can be difficult to image due to variable sample thickness, among other reasons. To address these issues, cell transfer engines based on “track etched” filter membrane technology have been used to prepare a more consistent single layer of cells that can be applied to a slide, analyzed and imaged. One known automated slide preparation system that has successfully utilized track etched membrane filters is the ThinPrep® 3000 system available from Cytyc Corporation 250 Campus Drive, Marlborough, Mass. 01752. The test using this system is generally referred to as a ThinPrep (TP) Papanicolaou (Pap) test, or more generally, a ThinPrep or TP test.

Referring to FIGS. 1 and 2, one known ThinPrep processing system 10 includes a container or vial 12 that holds a cytological specimen 14, a filter 20, a valve 30, and a vacuum source 40. The specimen 14 typically includes multiple cells 16 dispersed within a liquid, solution, fluid or transport medium 18, such as PreservCyt, also available from Cytyc Corporation. One known filter 20 includes filter material made of polyethylene terephthalate and has an average pore density of about 180,000 pores per square centimeter, an average pore diameter of about 6.9 microns, and an average membrane thickness of about 16 microns.

During use, one end of the filter 20 is inserted into the solution 18, and the other end of the filter 20 is coupled through the valve 30 to the vacuum source 40. When the valve 20 is opened, negative pressure from the vacuum source 40 is applied to the filter 20 which, in turn, draws solution 18 up into the filter 20. Cells 16 in the drawn liquid 18 are collected on the face of the filter 20. Referring to FIG. 3, the filter 20 having collected cells 16 is brought into contact with a slide 50. Referring to FIG. 4, the filter 20 is then removed from the slide 50, thereby preparing a specimen slide having a layer of cells 16.

While cell transfer engines based on “track etched” filter membrane technology have been used with effectiveness and provide significant improvements over other known methods, such devices and methods can be improved. In particular, it can be difficult to control the placement and presentation of individual cells 16 on the face of the filter 20. This may present difficulties in controlling the placement and presentation of cells 16 as they are transferred onto the slide 50, thereby making imaging and analysis or testing of the cells more complicated and time consuming.

FIG. 5 illustrates an example of a typical cell distribution and layout 60 of a specimen sample 14 prepared using “track etched” filter membranes, e.g., using a ThinPrep system. As shown in FIG. 5, and with further reference to FIG. 6, certain cells 16 may be grouped together to form a cluster or overlapping cells 17. Overlapping cells 17 may preclude the ability to determine cell 16 boundaries, generally illustrated in FIG. 7, with currently available imaging processing systems and techniques.

The ability to determine cell 16 boundaries is important since it allows full cell 16 border definition and the ability to obtain related cellular measurements and data such as cytoplasm area. These capabilities, in turn, allow accurate measurements of an important manual classification metric, namely, the nucleus/cytoplasm ratio which is an important cytological analysis parameter, and which has not been automatically measured in the past.

Further, membrane-based filters 20 do not allow for effective sorting of cells 16 or clusters 17 of cells by size. Additionally, while known preparation systems can be used to prepare specimens that can be stained, such systems may require relatively large volumes of stain and associated cumbersome staining equipment.

Microfluidics has been used recently to manipulate cells. Known microfluidic cell trapping techniques are described in “Cell trapping in Microfluidic chips,” by Robert M. Johann and “Single-Cell Enzyme Concentrations, Kinetics, and Inhibition Analysis Using High-Density Hydrodynamic Cell Isolation Arrays,” by Dino Di Carlo et al. and “Dynamic Single Culture Array” by Dino Di Carlo et al., the contents of all of which are incorporated herein by reference. Johannn describes various immobilization methods including contactless cell trapping and contact-based cell trapping. Di Carlo et al. describe a specific physical barrier that is designed to catch cells based on fluid flowing over an array of cell traps. Other microfluidics systems relate to detecting the presence of certain molecules, e.g., DNA.

While certain microfluidic devices and associated cell manipulation have been proposed, known microfluidic devices and techniques do not provide for effective separation, placement and transfer of cells from a heterogeneous sample of cells that includes other constituents such as lubricants and bodily fluids including blood and mucus. Further, known microfluidic devices do not provide these capabilities on a large scale to provide efficient specimen processing, including preparation and imaging of non-living, preserved specimen samples that are fixed to a substrate for purposes of examination and analysis. Therefore, known microfluidic devices and research are not suitable for cervical cytology and related preparation and analysis of such specimens.

### SUMMARY

According to one embodiment, a microfluidic apparatus for isolating cells of a cytological specimen includes a substrate and a microfluidic cellular isolation element associated with the substrate. The isolation element includes an outer wall, a channel, a partition member and a receptacle. The outer wall defines an inlet, an outlet, and an isolation element interior, and the channel is defined within the isolation element interior and in fluid communication with the outer wall inlet. The partition member is positioned within the isolation element interior and includes an inner wall that defines an inlet aperture, an outlet aperture, and a partition member interior. The receptacle is positioned within the partition member interior. The isolation element is configured such that fluid introduced through the outer wall inlet flows through the channel in a first direction, the partition member situated such that fluid flows from the channel into the partition member interior through the partition member inlet aperture in a second direction different than the first direction, the receptacle positioned relative to the partition member inlet to catch and retain a cell carried by the fluid.

According to another embodiment, a microfluidic apparatus for isolating cells of a cytological specimen includes a substrate and a microfluidic cellular isolation element associated with the substrate. The isolation element and the substrate are removably attached to each other. The isolation element includes an outer wall, a channel, a partition member and a receptacle. The outer wall defines an inlet, an outlet, and an isolation element interior, and the channel is defined within the isolation element interior and in fluid communication with the outer wall inlet. The partition member is positioned within the isolation element interior and includes an inner wall that defines an inlet aperture, an outlet aperture, and a partition member interior. The receptacle is positioned within the partition member interior. The isolation element configured such that fluid introduced through the outer wall inlet flows through the channel in a first direction, and the partition member situated such that fluid flows from the channel into the partition member interior through the partition member inlet aperture in a second direction different than the first direction. The receptacle is positioned relative to the partition member inlet to catch and retain a cell carried by the fluid. The isolation element and the substrate are removably attached to each other, and a cell caught and retained by the receptacle is located between the substrate and the isolation element.

A further embodiment is directed to a microfluidic apparatus for isolating cells of a cytological specimen that includes a substrate and a microfluidic cellular isolation element associated with the substrate. The isolation element includes an outer wall, a channel, a partition member and a plurality of receptacles. The outer wall defines an inlet, an outlet, and an isolation element interior, and the channel is defined within the isolation element interior and is in fluid communication with the outer wall inlet. The partition member is positioned within the isolation element interior and includes an inner wall that defines a plurality of inlet apertures, an outlet aperture, and a partition member interior, and the plurality of receptacles are situated within the partition member interior. Each receptacle includes a plurality of receptacle components that are separated from each other and arranged to catch a single cell or a cell cluster. The isolation element is configured such that fluid introduced through the outer wall inlet flows through the channel in a first direction, and the partition member situated such that fluid flows from the channel into the partition member interior through the respective partition member inlet apertures in a second direction different than the first direction. The receptacles are positioned relative to the partition member inlet apertures to catch and retain cells carried by the fluid.

A further alternative embodiment is directed to a method of isolating cells of a cytological specimen utilizing a microfluidic cellular isolation element associated with a substrate. The method includes introducing a fluid or solution through an inlet of an outer wall of the isolation element. The introduced fluid flows in a first direction through a channel defined within an interior or inner space of the isolation element. Fluid flows from the channel and through an inlet aperture of a partition member positioned within the isolation element interior in a second direction different than the first direction. The method also includes catching and retaining a cell carried by fluid flowing in the second direction in a receptacle positioned within the partition member. [0017] Another alternative embodiment is directed to a method of isolating and analyzing a cell of a cytological specimen utilizing a microfluidic cellular isolation element associated a substrate. The method includes introducing a fluid or solution through an inlet of an outer wall of the isolation element. The introduced fluid flows in a first direction through a channel defined within the interior or inner space of the isolation element. Fluid flows from the channel through an inlet aperture of a partition member positioned within the isolation element interior in a second direction different than the first direction. The method further includes catching and retaining a first cell in fluid flowing in the second direction in a receptacle positioned within the partition member. The method further includes releasing the first cell (e.g., after analyzing the first cell), and then catching and retaining a second cell in fluid flowing in the second direction to replace the released first cell. The second cell may then be analyzed.

Another alternative embodiment is directed to a method of isolating and imaging a cell of a cytological specimen utilizing a microfluidic cellular isolation element associated with a substrate. The method includes introducing a fluid or solution through an inlet of an outer wall of the isolation element. The introduced fluid flows in a first direction through a channel defined within the interior or inner space of the isolation element. Fluid flows from the channel through an inlet aperture of a partition member positioned within the isolation element interior in a second direction different than the first direction. The method further includes catching and retaining a first cell in fluid flowing in the second direction in a receptacle positioned within the partition member. The method further includes releasing the first cell from the receptacle (e.g., after processing or imaging the first cell), and then catching and retaining a second cell in fluid flowing in the second direction to replace the released first cell. The second cell may then be imaged.

In one or more embodiments, the second direction is substantially transverse to the first direction. Further, in one or more embodiments, a partition member includes multiple receptacles, and the receptacles may be different sizes. A smaller receptacle may be configured to catch and retain a single cell, and a larger receptacle may be configured to catch and retain a cluster of cells. In one embodiment, the smaller receptacle is positioned closer to the outer wall inlet than the larger receptacle. The inlet apertures of the partition member may also be different sizes. A smaller aperture may be configured to allow passage of a single cell, and a larger receptacle sized to allow passage of a cluster of cells. In one embodiment, the smaller inlet aperture is positioned closer to the outer wall inlet than the larger inlet aperture.

In one or more embodiments, an isolation element may include multiple partition members, thereby defining multiple channels, each of which is in fluid communication with the outer wall inlet, and at least one channel being defined between walls of adjacent partition members.

In one or more embodiments, a preconditioning element, e.g., located outside of the isolation element, configured to break apart cell clusters carried in the fluid. The cells and/or remaining clusters may then be caught by one or more receptacles within the isolation element.

Other and further aspects and embodiments are described herein and will become apparent upon review of the following detailed description and drawings.

### BRIEF DESCRIPTION OF THE DRAWINGS

Referring now to the drawings in which like reference numbers represent corresponding parts throughout and in which:

FIG. 1 illustrates a known slide preparation system and method that use a cytological membrane filter for collecting cells and applying a layer of collected cells to a specimen slide;

FIG. 2 is a bottom view of a known cytological membrane filter that includes collected cells to be applied to a specimen slide;

FIG. 3 illustrates a known method of applying cells collected by a cytological membrane filter to a specimen slide;

FIG. 4 shows a specimen slide having a layer of cells applied by a cytological membrane filter;

FIG. 5 illustrates an example of cell distribution on a specimen slide prepared using the type of system and method shown in FIGS. 1-4;

FIG. 6 further illustrates overlapping cells shown in FIG. 5;

FIG. 7 illustrates separated or isolated cells having defined boundaries;

FIG. 8A illustrates a microfluidic cellular isolation apparatus constructed according to one embodiment;

FIG. 8B illustrates a microfluidic cellular isolation apparatus constructed according to one embodiment in which a substrate and an isolation element are attached or adhered to each other;

FIG. 8C shows one embodiment involving removal of an isolation element from a substrate to form a substrate having isolated cells;

FIG. 8D further illustrates a substrate having isolated cells following removal of the isolation element as shown in FIG. 8C;

FIG. 8E shows one embodiment in which a cover slip applied over isolated cells on a substrate;

FIG. 9 illustrates a partition member and associated flows of solution within a microfluidic cellular isolation apparatus constructed according to one embodiment;

FIG. 10 illustrates a cell in solution approaching a cell receptacle within a partition member;

FIG. 11 illustrates a cell captured by the receptacle shown in FIG. 10;

FIG. 12 illustrates a cell receptacle constructed according to one embodiment and having two receptacle components;

FIG. 13 illustrates a cell receptacle constructed according to another embodiment and having three receptacle components;

FIG. 14 illustrates a microfluidic cellular isolation apparatus including a partition member having a plurality of inlet apertures and receptacles configured for catching individual cells according to a further embodiment;

FIG. 15 illustrates a microfluidic cellular isolation apparatus including a partition member having a plurality of larger inlet apertures and larger receptacles configured for catching clusters of cells according to a further embodiment;

FIG. 16 illustrates a microfluidic cellular isolation apparatus including multiple partition members and a plurality of inlet apertures and receptacles configured for catching individual cells according to another embodiment;

FIG. 17 illustrates a microfluidic cellular isolation apparatus including multiple partition members and a plurality of larger inlet apertures and larger receptacles configured for catching clusters of cells according to another embodiment;

FIG. 18 illustrates a microfluidic cellular isolation apparatus including multiple partition members and a plurality of inlet apertures and receptacles of different sizes for catching individual cells and clusters of cells according to another embodiment;

FIG. 19 illustrates the microfluidic cellular isolation apparatus shown in FIG. 18 including a preconditioning or disaggregation element according to another embodiment;

FIG. 20 illustrates a preconditioning element according to one embodiment; and

FIG. 21 generally illustrates a system that can be used for imaging cells and cell clusters.

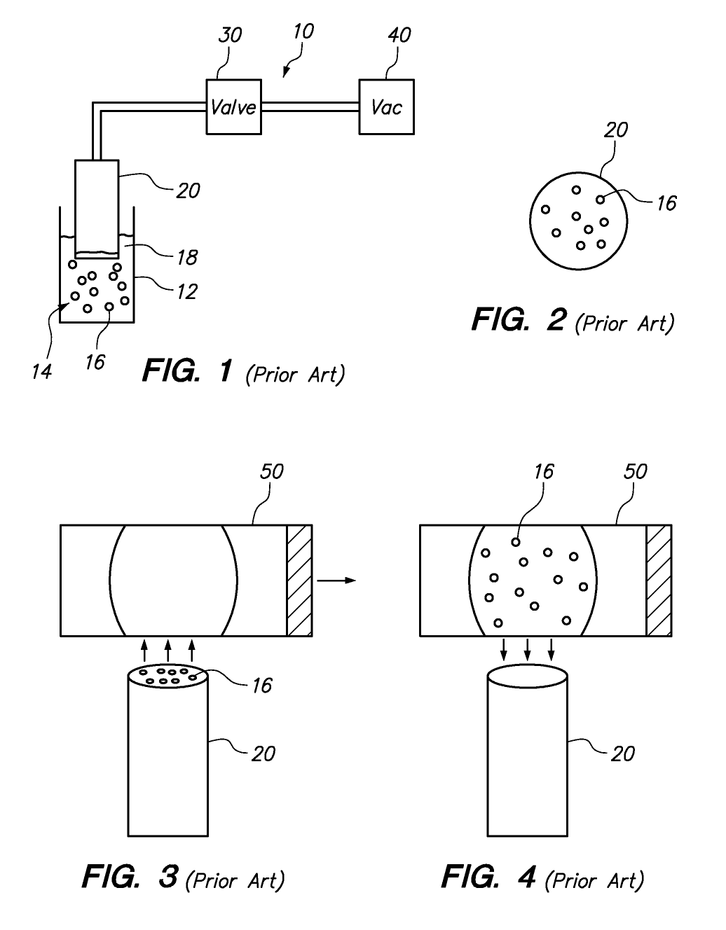

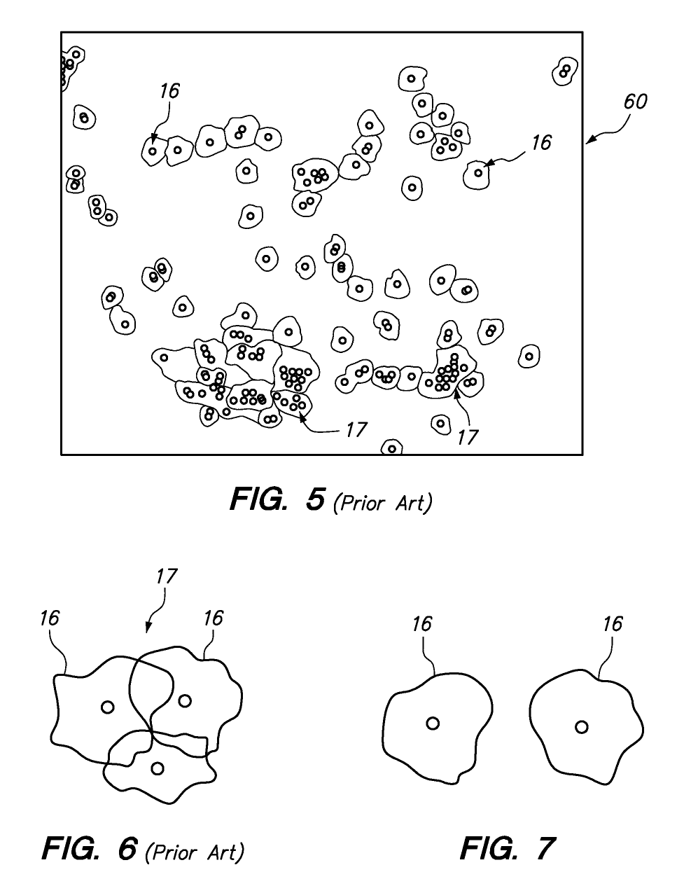

### DETAILED DESCRIPTION OF ILLUSTRATED EMBODIMENTS

In the following description, reference is made to the accompanying drawings which form a part hereof, and which show by way of illustration specific embodiments and how they may be practiced. It is to be understood that changes may be made without departing from the scope of embodiments.

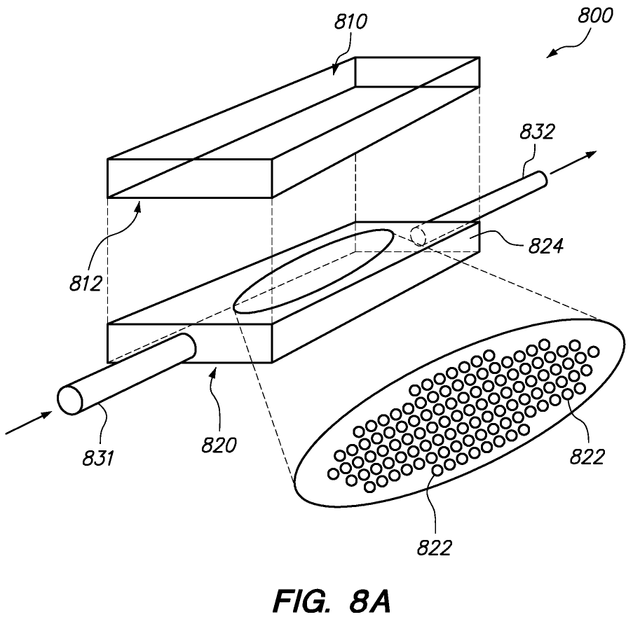

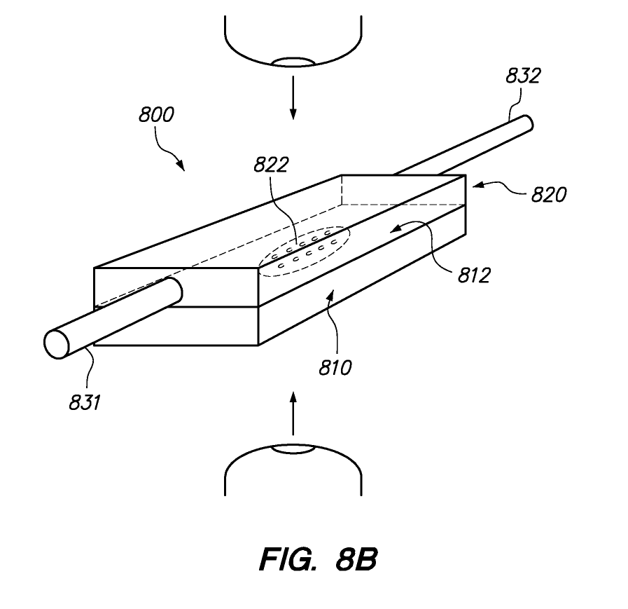

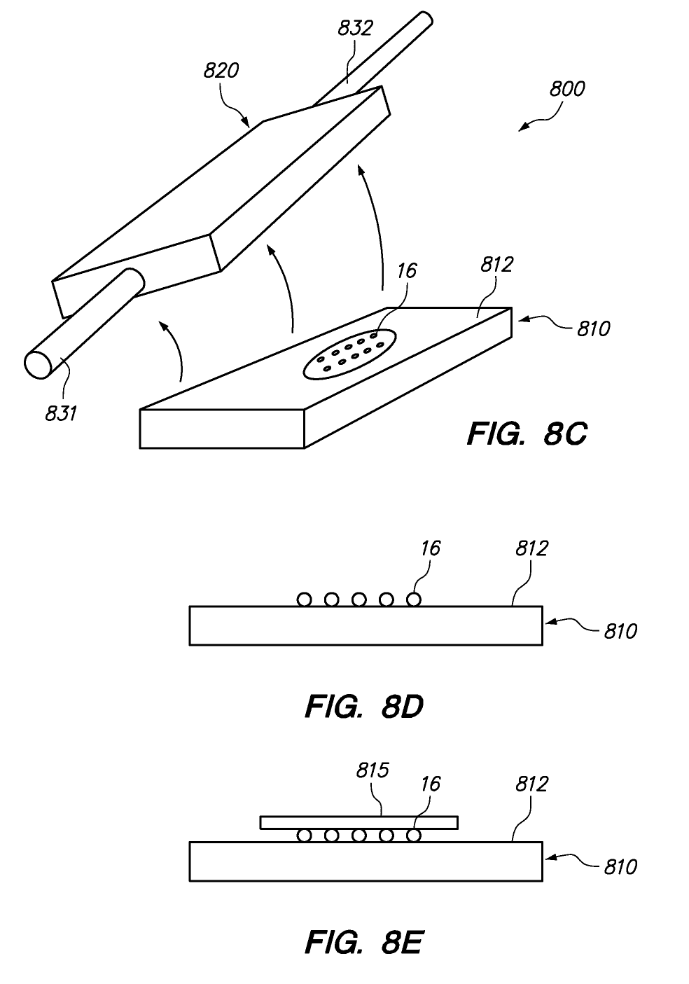

Referring to FIG. 8A, a microfluidic apparatus 800 constructed according to one embodiment and configured for isolating individual cells 16 and/or clusters 17 of cells of a cytological specimen includes a substrate or base member 810 (generally referred to as substrate 810), a microfluidic cellular isolation element 820 (generally referred to as isolation element 820), and a fluid inlet or inlet tube 831 (generally referred to as fluid inlet 831), a fluid outlet or outlet tube 832 (generally referred to as fluid outlet 832), or a fluid manifold (not shown in FIG. 8A). The microfluidic apparatus 800 is configured so that fluid or solution 18 flows through the micro-fabricated isolation element 820 in different directions in order to isolate cells 16 and/or clusters 17 of cells of a cytological specimen 14, thereby providing enhanced cell preparation, transfer, presentation and imaging. The fluid inlet and outlet 831, 832 are arranged to introduce solution 18 to be processed to the isolation element 820, and to remove processed solution 18 from the isolation element 820.

Embodiments may be used to isolate cells 16 and/or clusters 17 of cells, and reference is made generally to cells 16 unless certain configurations specifically involve isolation of clusters 17 of cells. Further, a solution or fluid as used in this specification is defined as a solution, fluid, material or substance that includes cells 16 or clusters 17 of cells and can flow through the microfluidic cellular isolation element 820. Examples of solutions or fluids 18 that may include cells 16 or clusters 17 of cells include a liquid-based solution, such as PreservCyt available from Cytyc, a gel-based solution, and a bodily fluid. Thus, cells 16 and clusters 17 of cells may be diluted in another substance or fluid (e.g., as in the case of a liquid-based or gel-based solution), or be part of a bodily fluid (e.g., cells of an undiluted specimen sample obtained directly from a cervix). For ease of explanation, reference is made to a solution 18 in the form of a liquid-based solution that flows through a microfluidic apparatus 800, but it should be understood that embodiments can be used to isolate cells in various solutions 18. Further, apparatus, system and method embodiments may be implemented to analyze cytological specimens including cervical specimens and other types of specimens. For ease of explanation, reference is made to cervical specimens in a solution 18.

In one embodiment, the substrate 810 is a glass substrate, such as a glass specimen slide. For example, the substrate 810 may be a known glass slide having a thickness of about 0.05 inch, a width of about 1.0 inch, and a length of about 3.0 inches. The substrate 810 may have a shape and size that is similar to or the same as a known specimen slide so that the substrate 810 can be manipulated by known slide processing systems and stored in known slide receptacles. In another embodiment, the apparatus 800 may be significantly smaller since the isolation element 820 has dimensions on the order of microns. Indeed other substrate dimensions and shapes can be utilized, and FIG. 8 is provided to generally illustrate a substrate 810 associated with an isolation element 820. For ease of explanation, reference is made to the substrate 810 being in the form of a known glass specimen slide.

As shown in FIG. 8A, the isolation element 820 is associated with the substrate 810, e.g., permanently or removably sealed, attached or adhered to a surface 812 or portion of substrate 810. According to one embodiment, the isolation element 820 is a polymer material, such as polydimethylsiloxane (PDMS). Other materials and polymer materials may also be utilized, and PDMS is provided as one example of a material that is suitable for micro-fabrication of components of the isolation element 820.

The isolation element 820 includes one or more cellular barriers, traps or partition members 822 (generally referred to as partition members 822) that are formed on or in PDMS material 824 using known micro-fabrication/micro-molding methods. The partition members 822 may include micro-fabricated channels, gates and cell receptacles or traps that are defined by the isolation element 820 and formed between the isolation element 820 and the surface 812 of the substrate 810 for catching and holding cells 16 from solution 18 that flows in different directions within the isolation element 820.

In the embodiment shown in FIG. 8A, the fluid inlet 831 and the fluid outlet 832 extend laterally from opposite sides of the isolation element 820. The fluid inlet 831 and fluid outlet 832 may also be formed of PDMS material 824 using known micro-fabrication/micro-molding methods. Thus, the isolation element 820, the partition members 822, the fluid inlet 831 and the fluid outlet 832 may be elements of an integrated, micro-molded or micro-fabricated component. The fluid inlet 831 is arranged to provide a solution 18 containing a cytological specimen 14 to the isolation element 820, which includes micro-fabricated partition members 822 that select, sort or isolate cells 16. The second or outflow tube 836 is arranged to carry solution 18 away from the isolation element 820 after the solution 18 has flowed through the isolation element 820.

The fluid inlet 831 and the fluid outlet 832 may also be utilized to introduce and carry away other fluids and solutions used for preparing or analyzing a cytological specimen 14. For example, the fluid inlet 831 and the fluid outlet 832 may serve as fluid path for cytological dyes or stains. Alternatively, separate inlet and outlet ports (not shown in FIG. 8A) may be provided or fabricated together with the isolation element 820 and fluid inlet and outlet 831, 832 for introducing and removing dyes and stains for staining collected cells 16. Embodiments advantageously provide “on-board” fluid handling for staining cells 16 with reduced volumes of stain and staining equipment of reduced size.

FIG. 8A illustrates one embodiment of an apparatus 800 in which the fluid inlet 831 and fluid outlet 832 are arranged horizontally or parallel to a plane of the isolation element 820. In alternative embodiments, the fluid inlet 831 and the fluid outlet 832 may be arranged vertically or at an angle relative to the isolation element 820 to provide solution to, and carry solution 18 from, the isolation element 820.

FIG. 8B illustrates one embodiment in which the glass substrate 810 and the isolation element 820 are attached or adhered together. FIG. 8B illustrates glass substrate 810 and isolation element 820 components that are rotated 180 degrees or flipped relative to the orientation shown in FIG. 8A so that the glass substrate 810 is shown on the bottom below the isolation element 820. Cells 16 captured by the partition members 822 are disposed between the glass substrate 810 and the isolation element 820. With this configuration, captured cells may be exposed to multiple stains and solutions by controlled flow of such stains and solutions through fluid inlet and fluid outlet 831, 832, or through separate input and output ports. A cytotechnologist may manipulate the assembly of the substrate 810 and the isolation element 820 to manually review and analyze the cells 16 by viewing the cells 16 through the substrate 810 or through the isolation element 820 since both of the glass substrate 810 and the isolation element (which may be PDMS) are transparent. Further, an automated slide processing system may image cells 16 by viewing the cells through one or both of the substrate 810 and isolation element 820 components, both of which may be transparent. With this configuration, a cover slip is not necessary since the cells 16 and/or clusters 17 of cells are sandwiched between the substrate 810 and the isolation element 820 and can be viewed directly without further preparation.

Referring to FIG. 8C, in an alternative embodiment, the micro-fabricated isolation element 820 and fluid inlet 831 and fluid outlet 832 extending from the isolation element 820 may be initially mated to a surface 812 of the substrate 810 so that cells 16 adhere to the glass surface 812. The isolation element 820 and the fluid inlet 831 and fluid outlet 832 extending there from may then be removed or peeled away. This results in cells 16 adhering to the surface 812 of the substrate 810, as shown in FIG. 8D. The cells may be additionally stained or treated as necessary, and referring to FIG. 8E, a cover slip 815 may be applied over the cells 16, which can then be imaged. It will be understood that dimensions of components and cells shown in various Figures may not accurately reflect actual or relative dimensions, and the sizes of components and cells are provided to illustrate how embodiments may be utilized.

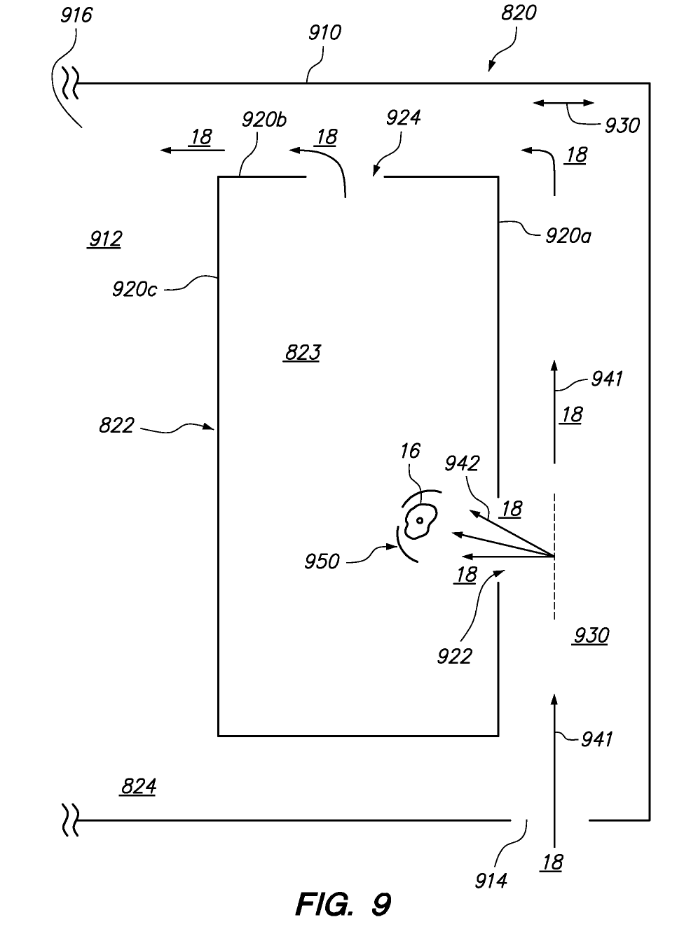

Referring to FIG. 9, a micro-fabricated isolation element 820 (or potion thereof) according to one embodiment is prepared using known micro-fabrication techniques and includes a micro-fabricated outer wall 910 and a micro-fabricated inner wall or partition member 822 (generally referred to as partition member 822). The outer wall 910 of the isolation element 820 defines an inner space or interior 912 in which the partition member 822 is formed, one or more fluid inlets 914 and one or more fluid outlet 916. The fluid inlet 914 is in fluid communication with a source of solution 18, e.g., the inflow tube 831, and the fluid outlet 916 is in fluid communication with, e.g., the outflow tube 832.

In the illustrated embodiment, the partition member 822 may be formed to have a rectangular shape, but the partition member 822 may have other shapes and configuration, e.g., square, triangular, diamond, and other shapes depending on the micro-fabrication technique and equipment that is utilized. FIG. 9 illustrates a generally rectangular partition member 822 to illustrate one example of how embodiments can be implemented using known micro-fabrication techniques and materials.

In the illustrated embodiment, the partition member 822 includes four sides 920a-d that define an inner space or interior 823. A first side 920a defines one or more inlet apertures or gates 922 (generally referred to inlet aperture 922). One inlet aperture 922 is shown for purposes of explanation, but it will be evident that the side 920a may define other numbers of inlet apertures 922. A top or downstream side 920b may define an outlet aperture 924. In the illustrated embodiment, the sides 920c and 920d are solid and do not define inlet or outlet apertures. Thus, in the illustrated embodiment, the partition member 822 may have a certain side 920a that defines only inlet apertures 922, a certain side 920b that defines only an outlet aperture 924, and certain sides 920c,d that are solid and define no apertures.

A micro-fabricated fluid channel 930 in fluid communication with the inlet 914, which is in fluid communication with the fluid inlet 831, is defined between the first side 920a of the partition member 822 and the outer wall 910. The base member 810 may have a thickness of about 6 mm, the isolation elements in 820 are molded within the base member 810, and the fluid channel 930 may be about 70 to about 100 microns wide, and about 40 microns in depth. In the illustrated example, the channel 930 extends around the corner of the partition member 822 defined by sides 920a,b. Solution 18 having cells 16 of the specimen 14 is introduced from the fluid inlet 831 and through the inlet 914. From the inlet 914, solution 18 flows downstream through the channel 930 along side 920a of the partition member 822.

The solution 18 initially flows through the channel in a first direction 941 (generally represented by an arrow parallel to the channel 930), otherwise referred to as laminar flow, or flow of solution 18 without turbulence. Laminar flow 941 within the channel 930 provides for the flow of solution 18 in a relatively predictable manner. A portion of the solution 18 flowing in the first direction 941 and through the channel 930 changes direction and flows through an inlet aperture 922 defined by the first side 920a of the partition member 822 (e.g., due to a pressure differential and/or surface adhesion). This is otherwise referred to as lateral flow, or flow in a second direction that is different than the first direction (generally represented by arrows that are not parallel to the arrow 941 or the channel 930). According to one embodiment, the isolation element 820 is fabricated so that the flow of solution 18 in the second direction 942 is substantially transverse or perpendicular to the laminar flow through the channel 930 in the first direction 941. According to one embodiment, the second direction 942 is at an angle of about 45 to 90 degrees relative to the first direction 941.

An individual cell 16 or a cluster 17 of cells carried by the solution 18 may be captured by a cell receptacle 950 positioned within the partition member 822 after the solution 18 flows through the inlet aperture 922 and towards the receptacle 950. For this purpose, the receptacle 950 may be arranged at a corresponding angle so that the open or receiving end of the receptacle 950 faces the inlet aperture 922 and is in the path of solution 18 flowing through the inlet aperture 922 in the second direction 942.

Solution 18 that enters the partition member 822 and flows past the receptacle 950 may continue to flow downstream through the interior 823 of the partition member 822, and exit the partition member 822 through the outlet aperture 924, where it may be re-combined with solution 18 that did not enter the partition member and flowed through the channel 930 around sides 920a,b. The solution 18 may then continue to flow downstream towards the outlet 916 and through the fluid outlet 832.

This micro-fabrication structure and the flow of solution 18 in different directions is beneficial since the lateral flow of solution 18 in the second direction 942 through the inlet aperture 922 minimizes clogging of the inlet apertures 922 by constituents such as lubricants and bodily fluids including blood and mucus. Further, the flow of solution 18 in the first direction 941 inhibits clogging of the inlet apertures 922 by flushing away particles that are too large to pass through the inlet apertures 922, while allowing particles (such as individual cells 16 or cell clusters 17) that are sufficiently small to pass through and traverse the inlet apertures 922 and lodge into a receptacle 950.

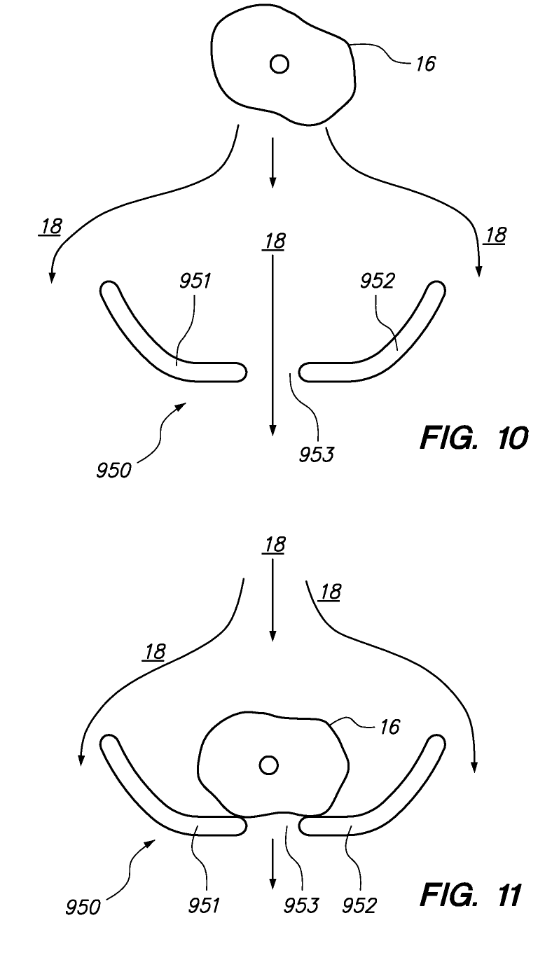

More particularly, as shown in FIG. 9, and with further reference to FIGS. 10 and 11, one embodiment includes a cell receptacle 950 that has a shape and size for holding a single cell 16. In the illustrated embodiment, the cell receptacle 950 includes two arcuate components 951, 952 that are separated from each other and arranged to form a “C” or “U” shaped structure and define a fluid passage 953 there between. Other embodiments of receptacles may be single component receptacles that do not define such fluid passages 953, however, reference is made to a receptacle 950 that does define a fluid passage 953 for ease of explanation

During use, solution 18 including cells 16 of a specimen 14 flows transversely in the second direction 942 through the inlet aperture 922 and towards the receptacle 950, which captures the cell 16 as shown in FIG. 11. Depending on the configuration of the receptacle 950, solution 18 may flow around the captured cell 16 and the receptacle 950 and exit the partition member 822 through the outlet aperture 924. A small amount of solution 18 may also flow through the passage 953 if the cell 16 does not completely block the passage 953, if the receptacle is so configured

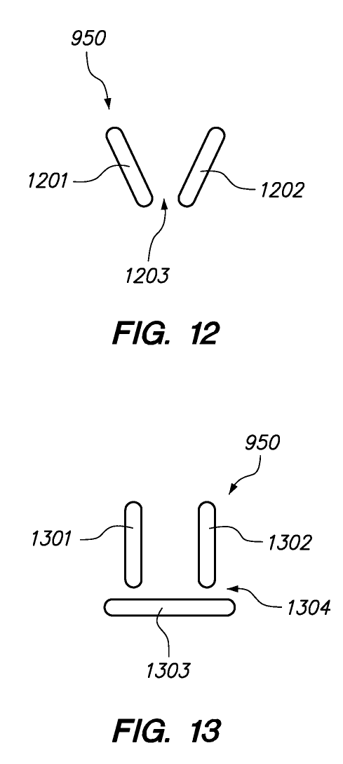

FIGS. 12 and 13 illustrate other receptacle 950 configurations that define a fluid passage and that may be used with embodiments to trap or capture individual cells 16 or clusters 17 of cells. In one embodiment, as shown in FIG. 12, a cell receptacle 950 includes two generally linear components 1201, 1202 that are arranged in a “V” shaped structure and define a fluid passage 1203 there between. FIG. 13 illustrates an alternative receptacle 950 that includes three components 1301, 1302, 1303. Two components 1301, 1302 are arranged substantially parallel to each other, and a third component 1303 is arranged substantially orthogonally to the other two components 1301, 1302 to define a fluid passage 1304 between the first and second components 1301, 1302 and the third component 1303. It should be understood, however, that other receptacle 950 configurations can be utilized; including receptacles 950 that do not define fluids passages such as fluid passages 1203 and 1304, and that receptacles 950 of different shapes, sizes and arrangements may be utilized to capture or trap individual cells 16 and cell clusters 17.

For example, a cell receptacle 950 as shown in FIGS. 9-11 may be configured for capturing an individual cell 16 and, for this purpose, defines an open or receiving end having a width or diameter of about 75 microns and a depth of about 25 microns. A cell receptacle 950 may also be configured for capturing a cluster 17 of cells and, for this purpose, may define an open or receiving end having a width or diameter of about 150 microns and a depth of about 50 microns. The configuration of the partition member 822, the number of inlet apertures 922 and number of receptacles 950 may also be selected to isolate different numbers and sizes of individual cells 16 and/or cell clusters 17. Thus, embodiments can advantageously be utilized to isolate an array of cells 16, an array of clusters 17, or an array of a mixture of individual cells 16 and clusters 17, which can then be processed further, e.g., stained and imaged as necessary.

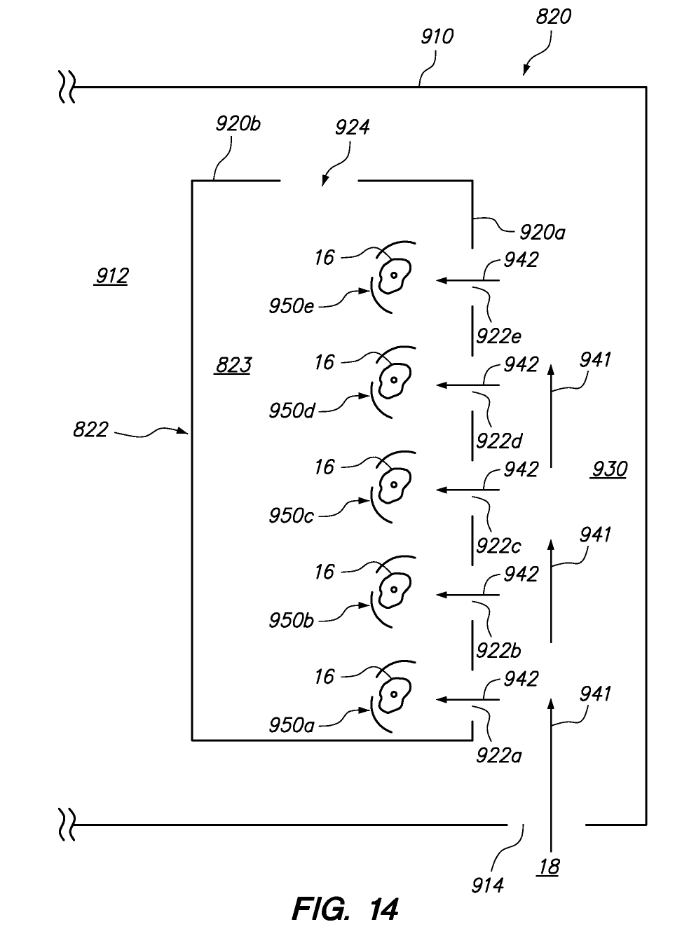

Referring to FIG. 14, according to one embodiment, the partition member 822 defines a plurality of inlet apertures 922a-e (generally 922) and a plurality of cell receptacles 950a-e (generally 950). Five receptacles 950 are shown for purposes of illustration, but it should be understood that a partition member 822 can have various numbers of receptacles 950, e.g., hundreds and thousands of such receptacles 950. In the illustrated embodiment, each cell receptacle 950 has a shape and size for capturing an individual cell 16. For this purpose, and given the particular arrangement of the inlet apertures 922 defined by one side 920a of the partition member 822, all of the cell receptacles 950 may face the same direction, i.e., towards corresponding inlet apertures 922. For example, solution 18 can flow in the first direction 941 through the channel 930 between the first side 920a of the partition member 822 and the outer wall 910, and then change direction and flow substantially transversely in the second direction 942 through inlet apertures 922a-e, thereby allowing individual cells 16 to be captured by respective receptacles 950a-e.

One example of a partition member 822 constructed in accordance with one embodiment for isolating individual cells 16 includes about 100 inlet apertures 922 and about 500 receptacles 950 within the partition member 822. The partition member 822 may have a width of about 500 microns and a height of several millimeters. Each inlet aperture 922 may have a width of about 75-200 microns, and the spacing between inlet apertures 922 may be about 100 microns. It should be understood that various other numbers and configurations of inlet apertures 922, receptacles 950 and partition members 822 may be utilized and may vary as necessary to capture a desired number of individual cells 16.

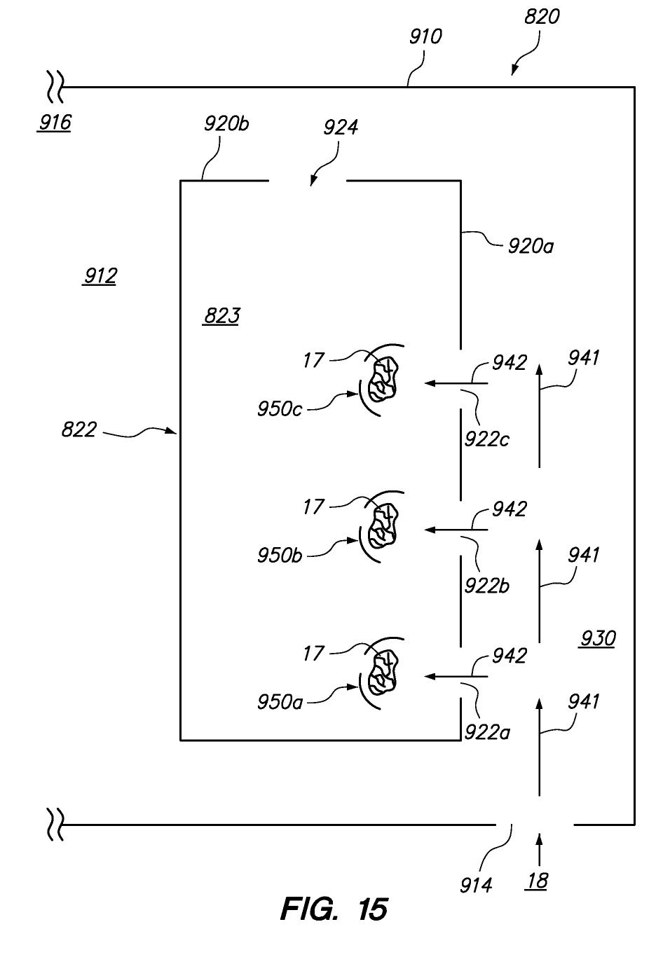

Referring to FIG. 15, according to another embodiment, the partition member 822 defines a plurality of inlet apertures 922a-c (generally 922) and a plurality of cell receptacles 950a-c (generally 950) having a shape and a size for capturing clusters 17 of individual cells. Three receptacles 950 are shown for purposes of illustration, and it should be understood that a partition member 822 can have various numbers of receptacles 950, e.g., hundreds and thousands of such receptacles 950. For this purpose, and given the particular arrangement of the inlet apertures 922 defined by one side 920a of the partition member 822, all of the cell receptacles 950 may face the same direction, i.e., towards a corresponding inlet aperture 922.

One example of a partition member 822 constructed in accordance with an embodiment for isolating clusters 17 of cells includes about 25 inlet apertures 922 and about 125 receptacles 950 within the partition member 822. The partition member 822 may have a width of about 500 microns, a height of several millimeters, each inlet aperture 922 may have width of about 200 microns, and the spacing between inlet apertures 922 may be about 100 microns. It should be understood that various other numbers and configurations of inlet apertures 922, receptacles 950 and partition members 822 may be utilized and may vary as necessary to capture a desired number of clusters 17.

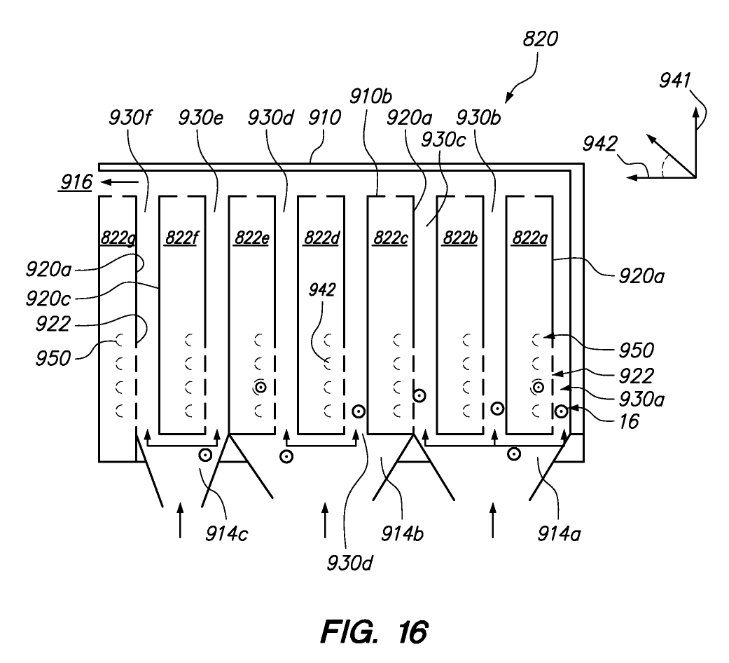

In other embodiments, an isolation element 820 may include multiple partition members, e.g., multiple partition members arranged side-by-side in an array with corresponding sides 920a-d. With this configuration, a corresponding array of cells 16 and/or clusters 17 of cells may be captured and processed. For example, referring to FIG. 16, an isolation member 820 constructed in accordance with another embodiment includes a plurality of partition members 822-g (generally 822), each of which is includes inlet apertures 922 and cell receptacles 950 configured for capturing individual cells 16. In the illustrated embodiment, the outer wall 910 of the isolation element 820 includes a plurality of inlets 914a-c (generally 914) for introducing solution 18 to different channels 930 (930a-f) (generally 930).

In the illustrated configuration, inlet apertures 922 are formed on the same side 910a of each partition member 822. A first channel 930a is defined between the side 920a of the partition member 822a and the outer wall 910 (as shown in FIGS. 9, 14 and 15). Additional channels 930b-f are defined between a side 920a of a partition member 822 that includes inlet apertures 922 and an opposite side 920c of another partition member that does not include inlet apertures 922. For example, a channel 930f is defined between the right side 920a of the partition member 822g that includes inlet apertures 922 and the left side 920c of adjacent partition member 822f that does not include inlet apertures 922. The inlets 914a-c of the outer wall 910 can be configured to provide solution 18 to a single channel or multiple channels (as shown in FIG. 16). It should be understood that partition members 822 can include additional downstream inlet apertures 922. Further, different partition members 822 can have different numbers of inlet apertures 922 and receptacles 950.

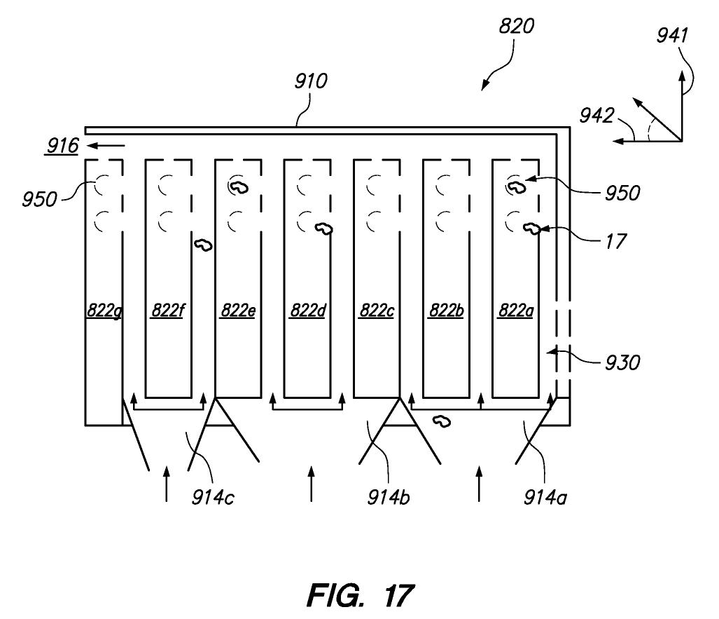

Referring to FIG. 17, an isolation member 820 constructed in accordance with another embodiment includes a plurality of partition members 822, each of which is includes inlet apertures 922 and cell receptacles 950 configured for capturing clusters 17 of cells. It should be understood that partition members 822 can include additional upstream inlet apertures 922. Further, different partition members can have different numbers of inlet apertures 922 and receptacles 950.

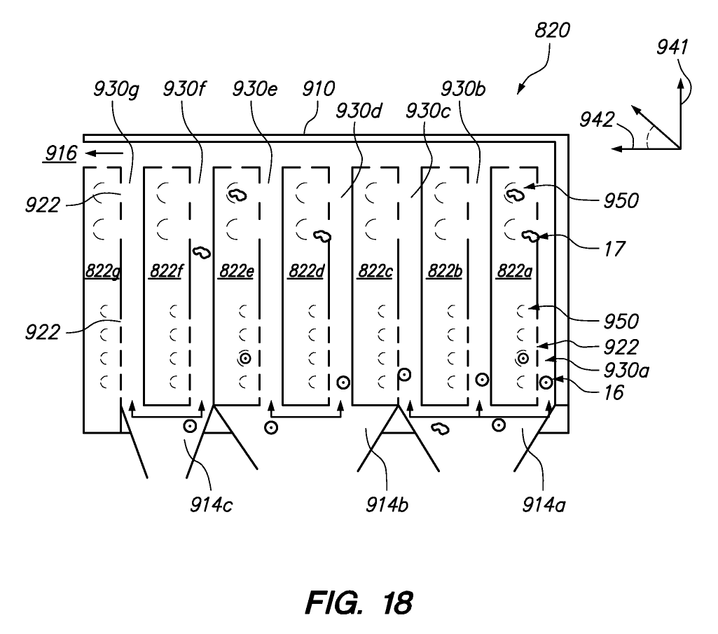

FIG. 18 illustrates another alternative embodiment of an isolation element 820 that includes multiple partition members 822 configured for capturing both individual cells 16 and clusters 17 of cells. In this embodiment, a partition member 822 includes inlet apertures 922 (upstream apertures) and receptacles 950 configured for passing and catching individual cells 16 that flow through the inlet apertures 922. A partition member 822 also includes larger inlet apertures 922 (downstream apertures) and receptacles 950 that are further away from the inlet 914 relative to the smaller inlet apertures 922 and respective receptacles 950. The larger inlet apertures 922 are configured for passing clusters 17 of cells, and receptacles 950 are positioned inside the partition member 822 to capture cell clusters 17 that flow through the larger inlet apertures 922. In this manner, solution 18 containing cell clusters 17 that are too large to pass through smaller, upstream inlet apertures 922 continues to flow through a channel 930 in the first direction 941, and then flows substantially transversely in the second or lateral direction 942 through a larger, downstream inlet aperture 922 so that the cluster 17 may be captured by a larger downstream cell receptacle 950. This configuration advantageously allows both individual cells 16 and clusters 17 of cells to be isolated, imaged and analyzed using a single isolation element 820.

FIGS. 17-19 illustrate an isolation element 820 including a plurality of partition members 822, e.g., seven partition members 822. It should be understood, however, that an isolation element 820 may include various numbers of partition members 822, e.g., about 50 to about 400 partition members 822. Additionally, although FIGS. 17-19 illustrate partition members 822 arranged in a single row, other arrangements may also e utilized, e.g., multiple rows, a column, a staggered configuration, etc.). Accordingly, FIGS. 17-19 are provided to generally illustrate different fluid flows and how cells 16 and clusters 17 may be captured using fluid flows in different directions.

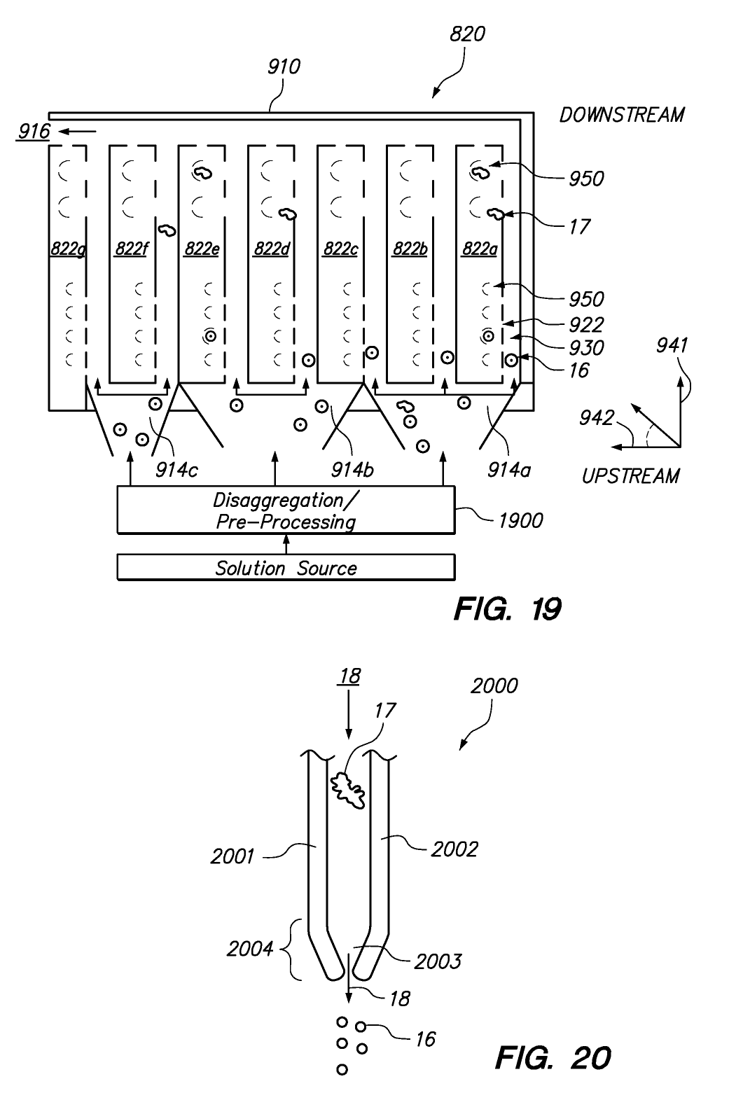

Referring to FIG. 19, if necessary, one or more micro-scale or macro-scale pre-processing chambers 1900 may positioned upstream of the isolation element 820, but downstream of the solution 18 source, such as the fluid inlet 831, in order to prepare or precondition solutions 18 for cellular isolation. For example, cell solutions 18 may be preconditioned by disaggregating large clusters 17 of cells by subjecting the clusters 17 to shear forces that are sufficient to separate or break apart the cluster 17 into smaller clusters 17 or into individual cells 16, without damaging individual cells 16. Alternatively, or additionally, lysed blood cell constituents and other debris may be removed by gates or filter elements having apertures or pore sizes that are smaller than the cells 16 and clusters 17. Further, combinations or sequences of dye solutions may be staged for staining purposes. For example, additional dedicated input and output ports (not shown) may be used for flowing dye over trapped cells 16 and clusters 17. Alternatively, the same fluid inlet 831 and fluid outlet 832 used for introducing solution 18 may also be used for introducing and removing stain and dye solutions, e.g., using a suitable a fluid junction mechanism).

According to one embodiment, referring to FIG. 20, a preconditioning element 2000 is generally in the form of a tapered tube or syringe that includes a first member 2001 and a second member 2002 that are separated from each other to define a fluid passage 2003 that is sufficiently wide to permit solution 18 with cells 16 and/or smaller clusters 17 to pass through the passage 2003. In the illustrated embodiment, clusters 17 in solution 18 are subjected to sufficient forces when passing through a tapered region 2004 defined by distal ends of the members 2001, 2003, thereby breaking apart the cluster 17. The pre-processing chamber 1900 may include various numbers of preconditioning elements 2000. It should be understood that other preconditioning element configurations can also be utilized.

In a further embodiment, a cell 16 that is captured within a cell receptacle 950 may be released, and another cell 16 can be captured to replace the released cell. For example, cells 16 that were initially captured by receptacles 950 may be imaged and examined. A cytotechnologist may then determine that certain cells 16 may be abnormal or suspicious, in which case these cells 16 may be retained, whereas cells 16 that are determined to be normal can be released and replaced with other captured cells 16 for examination. In this manner, a larger number of cells 16 are reviewed to provide a more thorough and accurate analysis by releasing and replacing normal cells 16 with other cells 16 that may be abnormal or suspicious. Release of captured cells 16 can be carried out using known mechanical, optical or electronic techniques, e.g., as described in “Cell trapping in Microfluidic chips,” by Robert M. Johann, the contents of which were previously incorporated herein by reference.

Cells 16 and cell clusters 17 that are isolated using the isolation element 820 mated or attached to the substrate 810 may be directly imaged as is, i.e., without having to transfer collected cells 16 and clusters 17 to another substrate or specimen slide since the captured cells 16 and clusters 17 may be visible through the substrate 810 and/or the isolation element 820. This capability provides enhanced automated reviewing of specimen samples having full cell border definitions so that measurements (such as cytoplasm area) can be completed and allow key manual classification metrics (such as the nucleus/cytoplasm ratio) to be automatically measured. Alternatively, the collected cells 16 and clusters 17 may be transferred from the isolation element 820 to another substrate, such as a glass specimen slide, and the transferred cells 16 and clusters 17 may then be imaged using various known cytological imaging systems.

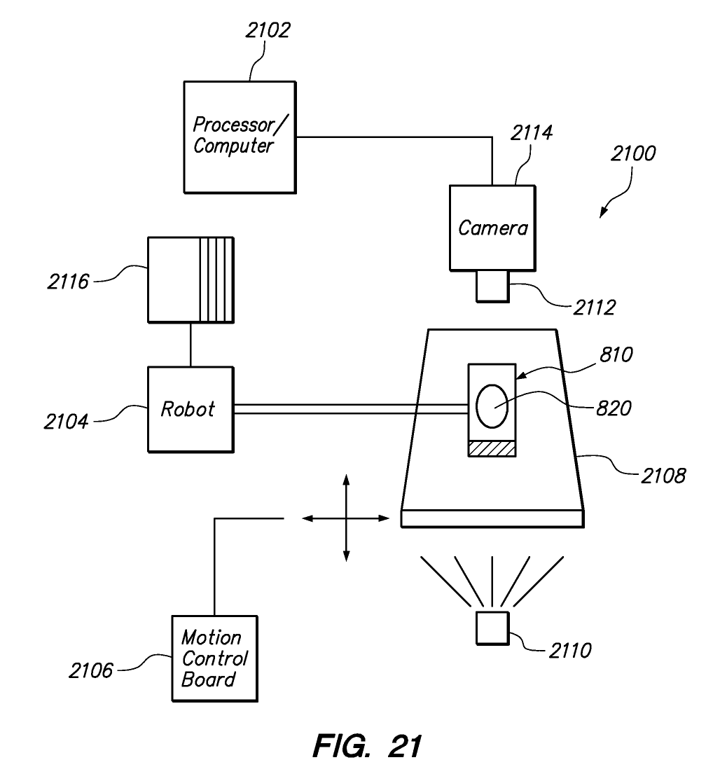

FIG. 21 generally illustrates an example of an imaging system 2110 that can be used to automatically acquire images of a specimen sample prepared utilizing isolation element embodiments. The system 2110 may be used in imaging applications in which collected cells 16 are not transferred to another substrate or slide (e.g., as shown in FIG. 8B), and when cells are transferred to a different carrier (e.g., as shown in FIGS. 8D-E).A typical imaging system 2100 includes a processor, computer or controller 2102, an optical stack, and a robot 2104. The optical stack includes a motion control board computer or controller 2106, a stage 2108, a light source 2110, a lens or a combination of optical elements as found in a microscope 2112 and a camera 2114. The robot 2104 may be configured for feeding and removing specimen samples including cells 16 or cell clusters 17 isolated by the isolation element 820 disposed on a substrate or glass slide 810 (assuming the substrate 810 is of a suitable size). The robot 2104 takes specimen sample from a cassette 2116 and places the slide 810 on the stage 2108. The computer 2102 controls the motion control board 2106 so that the motion control board 2106 moves the stage 2108 to locate the slide 810 under the camera 2114 and the lens 2112. The light source 2110 is activated, and an image of a portion of the specimen (i.e., one or more individual cells 16 or clusters 17) on the isolation element 820 disposed on the slide 810 is acquired by the camera 2114 and provided to the computer 2102. The computer 2102 instructs the motion control board computer 2106 to move the stage 2108 and the slide 810 thereon a very short distance from a first location to a second location. An image of the next portion of the specimen on the slide 810 (i.e., other cells 16 or clusters 17) at the second location is acquired by the camera 2114 and provided to the computer 2102. This process is repeated until all of the isolated cells and cell clusters are imaged. The robot 2104 then removes the imaged slide 810 from the stage 2108 and places another slide 810 from the cassette 2116 onto the stage 2108 for imaging as described above.

Images of the isolated cells 16 and cell clusters 17 generated by the optical stack are provided to the computer 2102 for analysis. After images of the isolated specimen cells 16 and cell clusters 17 are acquired, the images are processed to identify or rank cells and cell clusters that are of diagnostic interest. In some systems, this includes identifying those cells that most likely have attributes consistent with malignant or pre-malignant cells and their locations (x-y coordinates) on the slide. For example, the processor 2102 may select about 20 fields of view, e.g., 22 fields of view, which include x-y coordinates identifying the locations of cells 16 and cell clusters 17 that were selected by the processor 2101. This field of view or coordinate information is provided to a microscope, which steps through the identified x-y coordinates, placing the cells or clusters of cells within the field of view of the technician.

Although particular embodiments have been shown and described, it should be understood that the above discussion is not intended to limit the scope of these embodiments. Various changes and modifications may be made without departing from the spirit and scope of embodiments. For example, dimensions of various components are provided for purposes of explanation, and the sizes of components of embodiments may vary as necessary. Additionally, embodiments of microfluidic cell isolation devices may include various numbers of isolation elements, and each isolation element may include different numbers and configurations of partition members, inlet apertures, outlet apertures and cell receptacles. Further, embodiments may be implemented to capture only individual cells, only cell clusters, or a combination of individual cells and cell clusters as needed using cell receptacles of various shapes and sizes and having various numbers of receptacle elements. Embodiments may also be adapted or applied to isolating and analyzing cells of other types of specimens besides cervical specimens, and specimens may be in various solutions. Further, although embodiments are described with reference to micro-fabrication and hydrodynamics, embodiments may also be implemented using other microfluidic techniques including optical or dielectrophoretic techniques. Thus, embodiments are intended to cover alternatives, modifications, and equivalents that fall within the scope of the claims.

What is claimed is:

1. A microfluidic apparatus for isolating cells of a cytological specimen, comprising:
a substrate; and
a microfluidic cellular isolation element associated with the substrate, the isolation element comprising
an outer wall defining an inlet, an outlet, and an isolation element interior,
a channel defined within the isolation element interior and in fluid communication with the outer wall inlet,
a partition member positioned within the isolation element interior, the partition member comprising an inner wall defining an inlet aperture, an outlet aperture, and a partition member interior, and
a receptacle positioned within the partition member interior,
the isolation element configured such that fluid introduced through the outer wall inlet flows through the channel in a first direction, the partition member situated such that fluid flows from the channel into the partition member interior through the partition member inlet aperture in a second direction different than the first direction, the receptacle positioned relative to the partition member inlet to catch and retain a cell carried by the fluid.

2. The apparatus of claim 1, wherein the second direction is substantially transverse to the first direction.

3. The apparatus of claim 1, the first direction being substantially parallel to a lengthwise dimension of the inner wall.

4. The apparatus of claim 1, the isolation element comprising a polymer.

5. The apparatus of claim 1, the receptacle comprising a plurality of receptacle components separated from each other and arranged to catch an individual cell.

6. The apparatus of claim 1, the inner wall defining a plurality of inlet apertures, wherein a corresponding plurality of receptacles are situated within the partition member interior to respectively catch and retain one or more cells carried by fluid flowing from the channel into the partition member interior through the respective partition member inlet apertures.

7. The apparatus of claim 6, the receptacles facing the same direction.

8. The apparatus of claim 6, the receptacles being approximately the same size.

9. The apparatus of claim 6, the receptacles being different sizes.

10. The apparatus of claim 9, the receptacles including a smaller receptacle sized to catch and retain a single cell, and a larger receptacle sized to catch and retain a cluster of cells, the smaller receptacle being positioned closer to the outer wall inlet than the larger receptacle.

11. The apparatus of claim 6, the inlet apertures being approximately the same size.

12. The apparatus of claim 6, the inlet apertures being different sizes.

13. The apparatus of claim 12, the inlet apertures including a smaller aperture sized to allow passage of a single cell, and a larger receptacle sized to allow passage of a cluster of cells, the smaller inlet aperture being positioned closer to the outer wall inlet than the larger inlet aperture.

14. The apparatus of claim 1, the interior of the isolation element including a plurality of partition members and a plurality of channels, each channel in fluid communication with the outer wall inlet, with at least one channel being defined between walls of adjacent partition members.

15. The apparatus of claim 1, further comprising a preconditioning element located outside of the isolation element and configured to break apart cell clusters carried in the fluid.

16. The apparatus of claim 1, the isolation element removable from the substrate in a manner leaving a cell caught and retained by the receptacle on the substrate.

17. A microfluidic apparatus for isolating cells of a cytological specimen, comprising:
a substrate; and
a microfluidic cellular isolation element associated with the substrate, the isolation element comprising
an outer wall defining an inlet, an outlet, and an isolation element interior,
a channel defined within the isolation element interior and in fluid communication with the outer wall inlet,
a partition member positioned within the isolation element interior, the partition member comprising an inner wall defining an inlet aperture, an outlet aperture, and a partition member interior, and
a receptacle positioned within the partition member interior,
the isolation element configured such that fluid introduced through the outer wall inlet flows through the channel in a first direction, the partition member situated such that fluid flows from the channel into the partition member interior through the partition member inlet aperture in a second direction different than the first direction, the receptacle positioned relative to the partition member inlet to catch and retain a cell carried by the fluid,
the isolation element and the substrate being removably attached to each other such that a cell caught and retained by the receptacle is located between the substrate and the isolation element.

18. The apparatus of claim 17, wherein a cell caught and retained by the receptacle is viewable through at least one of the substrate and the isolation element.

19. A system including the apparatus of claim 17 and further comprising an imager configured to acquire an image of a cell caught and retained by the receptacle.

20. A microfluidic apparatus for isolating cells of a cytological specimen, comprising:
a substrate; and
a microfluidic cellular isolation element associated with the substrate, the isolation element comprising
an outer wall defining an inlet, an outlet, and an isolation element interior,
a channel defined within the isolation element interior and in fluid communication with the outer wall inlet,
a partition member positioned within the isolation element interior, the partition member comprising an inner wall defining a plurality of inlet apertures, an outlet aperture, and a partition member interior, and
a plurality of receptacles situated within the partition member interior, each receptacle comprising a plurality of receptacle components separated from each other and arranged to catch a single cell or a cell cluster,
the isolation element configured such that fluid introduced through the outer wall inlet flows through the channel in a first direction, the partition member situated such that fluid flows from the channel into the partition member interior through the respective partition member inlet apertures in a second direction different than the first direction, the receptacles positioned relative to the partition member inlet apertures to catch and retain cells carried by the fluid.

21. The apparatus of claim 20, wherein the second direction is substantially transverse to the first direction.

22. The apparatus of claim 20, the receptacles being different sizes, including a smaller receptacle sized to catch and retain a single cell, and a larger receptacle sized to catch and retain a cluster of cells, the smaller receptacle being positioned closer to the outer wall inlet than the larger receptacle.

23. The apparatus of claim 22, the inlet apertures being different sizes, including a smaller aperture sized to allow passage of a single cell, and a larger receptacle sized to allow passage of a cluster of cells, the smaller inlet aperture being positioned closer to the outer wall inlet than the larger inlet aperture.

24. The apparatus of claim 20, the interior of the isolation element including a plurality of partition members and a plurality of channels, each channel in fluid communication with the outer wall inlet, with at least one channel being defined between walls of adjacent partition members.

25. The apparatus of claim 20, further comprising a preconditioning element positioned outside of the isolation element and configured break apart cell clusters carried in the fluid.
